(19) United States
(12) Patent Application Publication
(10) Pub. No.: US 2021/0352395 A1
(43) Pub. Date: Nov. 11, 2021

(54) EARBUD

(71) Applicant: Merry Electronics(Shenzhen) Co., Ltd., ShenZhen (CN)

(72) Inventors: Ko Min Wang, Taichung (TW); Chung-Yi Huang, Taichung (TW)

(73) Assignee: Merry Electronics(Shenzhen) Co., Ltd., ShenZhen (CN)

(21) Appl. No.: 17/121,758

(22) Filed: Dec. 15, 2020

Related U.S. Application Data
(60) Provisional application No. 63/020,556, filed on May 6, 2020.

(51) Int. Cl. H04R 1/10; A61B 5/00

(52) U.S. Cl. CPC H04R 1/1016; A61B 5/6817

(57) ABSTRACT

An earbud including a main body, a sound guiding structure, a battery, and a sensor is provided. A geometric shape of the main body has a long axis and a short axis orthogonal to each other. When the earbud is worn, the main body is housed in a concha cavity of a user, and a tragus of the user and the long axis are located on a same level. The sound guiding structure extends away from the main body from the long axis of the main body. The battery and the sensor are disposed in the main body, and the sensor is adjacent to a periphery of the battery.

### CROSS-REFERENCE TO RELATED APPLICATION

This application claims the priority benefit of U.S. Provisional Application No. 63/020,556, filed on May 6, 2020. The entirety of the above-mentioned patent application is hereby incorporated by reference herein and made a part of this specification.

### BACKGROUND

### Technical Field

The disclosure relates to an earbud.

### Description of Related Art

Generally, in most of the wearable physiological measurement apparatuses, electrical signal measurement is employed. Nevertheless, the structure required by the electrical signal measurement is complicated, and such apparatuses may not be conveniently worn, preventing a user from performing detection at any time as a result. Such problem may be overcome by using an existing wearable physiological measurement apparatus adopting optical signal measurement.

Regarding such an apparatus, an optical signal measurement module is integrated into an earbud, so that the earbud may not only be used to listen to the music but also be used to monitor physiological signals such as heartbeat. At present, most of the optical signal measurement modules that may be integrated into the earbuds are configured to correspond to the lower edge of the user's ear, so only the skin surface is measured. Nevertheless, the skin on the outer ears of the human body is thin and thus may be easily penetrated by external environment light. Interference caused by the environment light may not be easily prevented, and inaccurate measurement is thereby provided.

Further, the volume of the optical signal measurement modules integrated into the earbuds is generally designed to be large to allow the modules to fit the concha cavities of the user as much as possible, so that light leakage may be prevented from occurring. Such design, however, may cause pressure and discomfort to the user's ears, and as such, the user may not enjoy a favorable wearing experience as a result. In other words, at present, the earbuds integrated with the optical signal measurement modules may neither be worn for a long time nor provide accurate measurement without being interfered.

### SUMMARY

The disclosure provides an earbud capable of providing a comfortable wearing experience required for long-term wearing and accuracy required for optical signal measuring.

The disclosure provides an earbud including a main body, a sound guiding structure, a battery, and a sensor. A geometric shape of the main body has a long axis and a short axis orthogonal to each other. When the earbud is worn, the main body is housed in a concha cavity of a user, and a tragus of the user and the long axis are located on a same level. The sound guiding structure extends away from the main body from the long axis of the main body. The battery and the sensor are disposed in the main body, and the sensor is adjacent to a periphery of the battery.

In an embodiment of the disclosure, a portion of the sensor protrudes from an inner side surface of the main body, the inner side surface faces a concha cavity wall of the user, and the sound guiding structure extends out from the inner side surface.

In an embodiment of the disclosure, the sound guiding structure and the sensor are located on two opposite ends of the long axis, and the battery is located between the sound guiding structure and the sensor.

In an embodiment of the disclosure, a position of the battery on the long axis and a position of the sensor on the long axis do not overlap with each other.

In an embodiment of the disclosure, the position of the sensor on the long axis and a position of the battery on the long axis at least partially overlap, and an overlap is away from the sound guiding structure.

In an embodiment of the disclosure, the sensor and the sound guiding structure are located on a same side of the battery, and a gap is provided between the battery and the sound guiding structure.

In an embodiment of the disclosure, the sensor is disposed on one side of the battery along the short axis and is located above the level.

In an embodiment of the disclosure, the sensor is disposed on one side of the battery along the short axis and is located below the level.

In an embodiment of the disclosure, the earbud further includes a speaker disposed in the sound guiding structure.

In an embodiment of the disclosure, the speaker is adjacent to the battery, and a position of the sensor on the long axis and a position of the battery on the long axis do not overlap with each other.

In an embodiment of the disclosure, a gap is provided between the speaker and the battery, and a position of the sensor on the long axis and a position of the battery on the long axis at least partially overlap.

In an embodiment of the disclosure, in a front view when the earbud is worn, an included angle is provided between an extending axis direction of the sound guiding structure and the long axis, and the included angle is 0 degrees to 45 degrees.

In an embodiment of the disclosure, in a top view when the earbud is worn, an included angle is provided between an extending axis direction of the sound guiding structure and the long axis, and the included angle is 45 degrees to 80 degrees.

In an embodiment of the disclosure, a ratio of the long axis to the short axis is: short axis/long axis=0.7 to 0.9.

In an embodiment of the disclosure, a length of the short axis is 13.5 mm to 15.5 mm.

To sum up, the main body of the earbud is integrated with the battery and the sensor. Herein, the long axis and the short axis may be identified on the geometric shape of the main body, so that the tragus of the user and the long axis may be configured to be located on the same level when the earbud is worn. The sound guiding structure extends away from the main body from the long axis of the main body. Through the above features, the earbud may be securely housed in the concha cavity of the user. Further, the sensor which is disposed in the main body together with the battery is adjacent to the periphery of the battery. Therefore, through the foregoing arrangement, the earbud may not only set the sensor and the battery to be housed in the main body but also accordingly form a compact arrangement among members, and structural assembly may thus be smoothly completed.

Accordingly, through the above-mentioned structural features and member arrangement, the earbud may provide the required optical signal measurement effect, and the user may at the same time enjoy a comfortable wearing experience when wearing the earbud, so that the earbud may be worn for a long time and hear rate may be sensed at any time.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A is a schematic view of an earbud according to an embodiment of the disclosure.

FIG. 1B illustrates the earbud of FIG. 1A from another viewing angle.

FIG. 2 is a schematic view illustrating the earbud being worn on the ear of a user.

FIG. 3A is a perspective schematic view illustrating the earbud of FIG. 2.

FIG. 3B is a top view illustrating the earbud of FIG. 2.

FIG. 4A is a top view of an earbud according to another embodiment of the disclosure.

FIG. 4B is a front view of the earbud of FIG. 4A.

FIG. 5 and FIG. 6 are front views of earbuds according to different embodiments.

### DESCRIPTION OF THE EMBODIMENTS

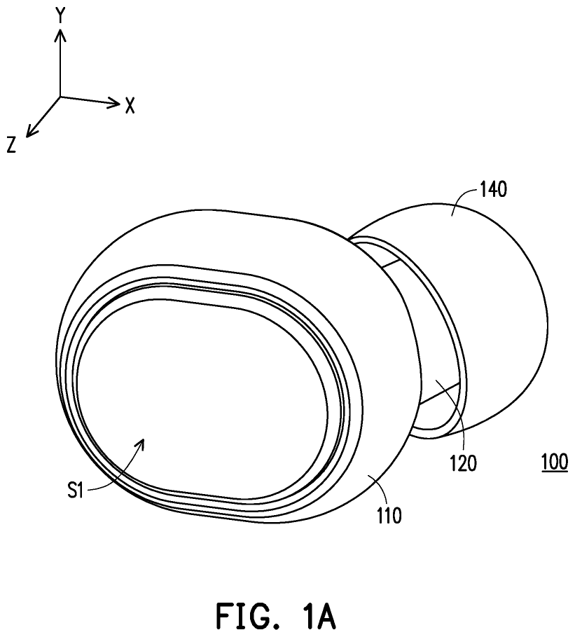

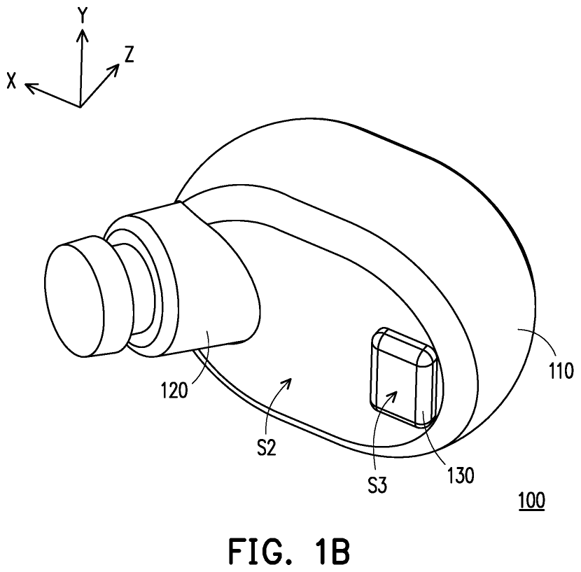

FIG. 1A is a schematic view of an earbud according to an embodiment of the disclosure. FIG. 1B illustrates the earbud of FIG. 1A from another viewing angle. FIG. 2 is a schematic view illustrating the earbud being worn on the ear of a user. A Cartesian coordinate system X-Y-Z is also provided in this embodiment for ease of description of members. With reference to FIG. 1A, FIG. 1B, and FIG. 2 together, in this embodiment, from an appearance of an earbud 100, a main body 110, a sound guiding structure 120, a sensor 130, and an ear sleeve 140 may be clearly identified. A geometric shape of the main body 110 has a long axis L and a short axis W orthogonal to each other. The sound guiding structure 120 extends away from the main body 110 from the long axis L of the main body 110. In this way, when the earbud 100 is worn, the main body 110 is housed in a concha cavity 21 of a user, and a tragus 22 of the user and the long axis L are located on a same level. As shown in FIG. 2, the tragus 22 is located in an extending axis direction of the long axis L, and the earbud 100 may thus be securely worn in the concha cavity 21 of the user without being detached. At the same time, one end of the short axis W of the main body 110 falls on the tragus 23, so that an outer surface S1 of the main body 110 may be securely attached by the tragus 22 at a position of the tragus 22, and a required secure clamping effect is thereby provided.

Note that the ear sleeve 140 provided by the present embodiment is illustrated in FIG. 1A merely and is omitted in following figures for ease of identification of other members.

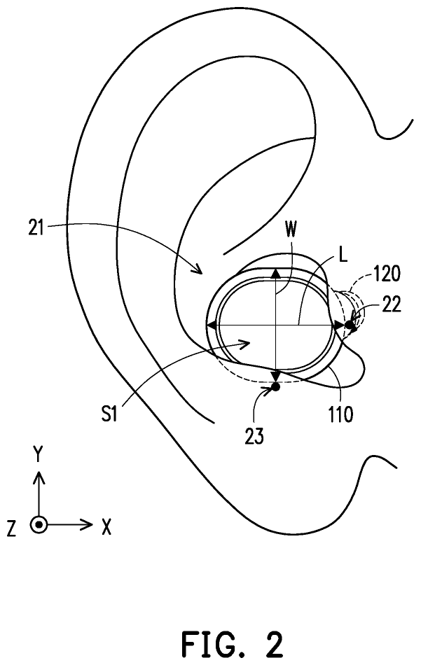

FIG. 3A is a perspective schematic view illustrating the earbud of FIG. 2. FIG. 3B is a top view illustrating the earbud of FIG. 2. With reference to FIG. 1B and FIG. 3B first, in this embodiment, a portion of the sensor 130 protrudes from an inner side surface S2 of the main body 110. That is, a sensing surface S3 of the sensor 130 protrudes from the inner side surface S2 of the main body 110. Further, the sound guiding structure 120 extends from the inner side surface S2, and the sound guiding structure 120 is shaped as a tube or has a shape structure with a nozzle. When the earbud 100 is worn, the inner side surface S2 faces a cavity inner wall of the concha cavity 21 of the user. That is, the sensing surface S3 of the sensor 130 is securely attached to a cavity wall of the concha cavity 21. In this way, optical sensing is performed on the cavity wall of the concha cavity 21 by the sensor 130, so heart rate of the user may thus be obtained. As such, the sensing surface S3 is allowed to face away from the outer surface S1 of the main body 110 and faces the ear, and interference caused by external environment light may thereby be effectively prevented. Further, since the cavity wall of the concha cavity 21 is smooth, the sensor 130 may accurately sense hear rate data of the user.

Besides, with reference to FIG. 2, FIG. 3A, and FIG. 3B together, in this embodiment, a length of the short axis W of the main body 110 is 13.5 mm to 15.5 mm, and a ration of the long axis L to the short axis W is: short axis W/long axis L=0.7 to 0.9. Further, in FIG. 3A, an included angle θ1 is provided between an extending axis direction of the sound guiding structure 120 and the long axis L, and the included angle θ1 is 45 degrees to 80 degrees. In FIG. 3B,80 an included angle θ2 is provided between the extending axis direction of the sound guiding structure 120 and the long axis L, and the included angle θ2 is 0 degrees to 45 degrees. Accordingly, an outer structure feature of the main body 110 of the earbud 100 and a corresponding relationship between related members and the main body 110 provided by the present embodiment are further defined. Further, in a front view when the earbud 100 is worn as shown in FIG. 3A, the included angle θ1 is provided between the extending axis direction of the sound guiding structure 120 and the long axis L. That is, on the X-Y plane where both the long axis L and the short axis W are located, θ1 is provided between an orthogonal projection of the sound guiding structure 120 on the X-Y plane and the long axis L located on the X-Y plane. Similarly, in a top view when the earbud 100 is worn as shown in FIG. 3B, the included angle θ2 is provided between the extending axis direction of the sound guiding structure 120 and the long axis L. That is, on the plane (X-Z plane) formed by a normal line (Z axis) of the X-Y plane and the long axis L, the included angle θ2 is provided between an orthogonal projection of the extending axis direction of the sound guiding structure 120 on the X-Z plane and the long axis L.

Further, with reference to FIG. 3A and FIG. 3B together, the earbud 100 provided by the present embodiment further includes a battery 150 and a speaker 160. The speaker 160 is disposed in the sound guiding structure 120, the battery 150 is disposed in the main body 110, and the sensor 130 is adjacent to a periphery of the battery 150. In other words, in this embodiment, arrangement of the inner members of the earbud 100 is further defined. First, the sound guiding structure 120 and the sensor 130 are configured to be located on two opposite ends of the long axis L, and the battery 150 is located between the sound guiding structure 120 and the sensor 130. Second, a position of the battery 150 on the long axis L and a position of the sensor 130 on the long axis L do not overlap with each other. Third, the speaker 160 is further configured to be adjacent to the battery 150. Accordingly, in a space formed by the main body 110 and the sound guiding structure 120 extending from the main body 110, the speaker 160, the battery 150, and the sensor 130 are substantially disposed along the long axis L in a compact manner. In other words, in the limited space (as shown in FIG. 2) provided by the concha cavity 21 of the user, the long axis L of the main body 110 of the earbud 100 and the tragus 22 are configured to be located on the same level as described above, and further in this embodiment, the related members may be well covered in the main body 110 and the sound guiding structure 120 through the above-mentioned compact arrangement.

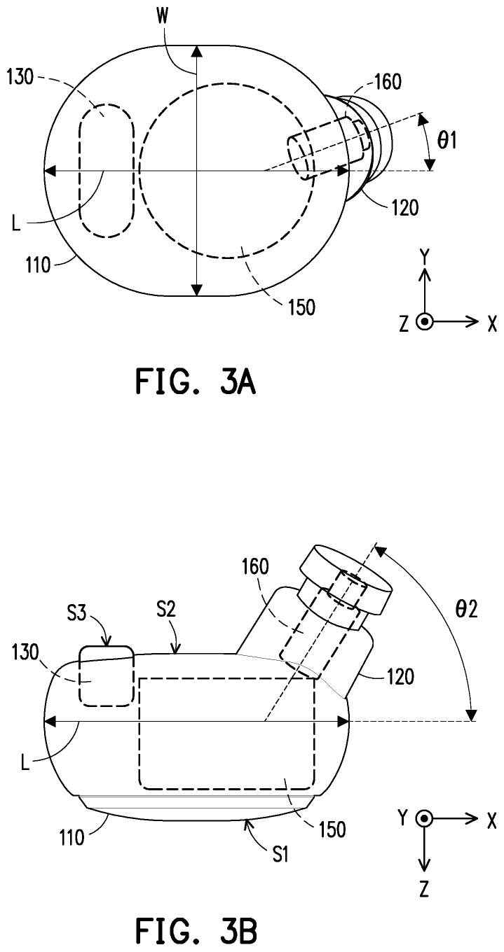

FIG. 4A is a top view of an earbud according to another embodiment of the disclosure. FIG. 4B is a front view of the earbud of FIG. 4A. With reference to FIG. 4A and FIG. 4B together, the same perspective manner is employed herein, so that internal members of an earbud 200 may be easily identified. Different from the foregoing embodiments, in the earbud 200, a position of the sensor 130 on the long axis L and a position of a battery 250 on the long axis L at least partially overlap, and an overlap is away from the sound guiding structure 120. Further, the sensor 130 and the sound guiding structure 120 are located on a same side of the battery 250, and a gap G1 is provided between the battery 250 and the sound guiding structure 120, which is equivalent to that the gap G1 is provided between the speaker 160 and the battery 250.

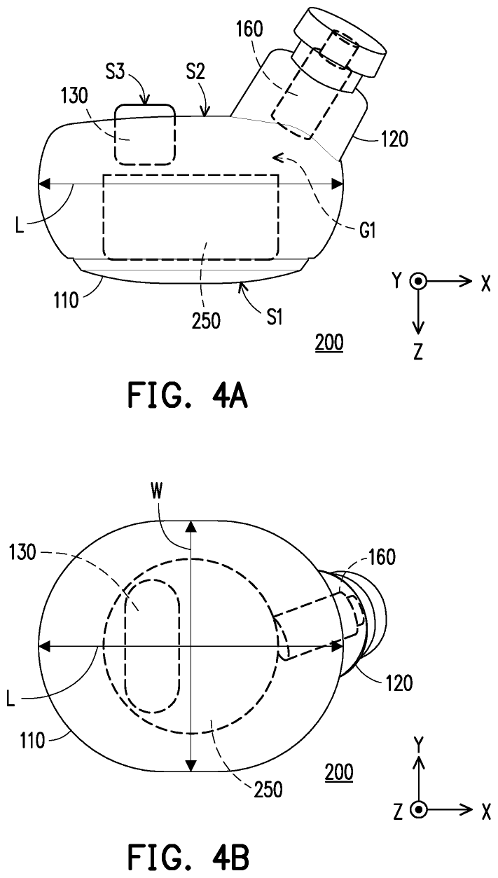

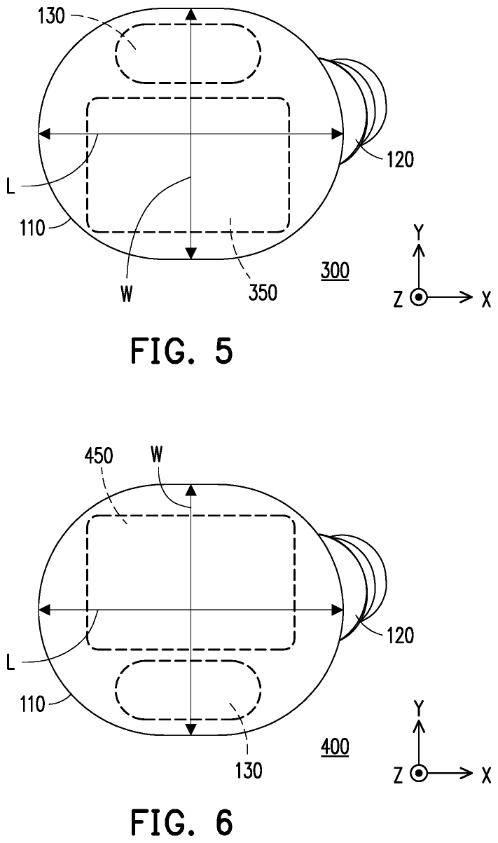

FIG. 5 and FIG. 6 are front views of earbuds according to different embodiments. With reference to FIG. 5 first together with FIG. 2, an earbud 300 provided the present embodiment may still be smoothly worn in the concha cavity 21 of the user, and the earbud 300 and the tragus 22 are located on the same level. Different from the foregoing embodiments, the sensor 130 provided by this embodiment is disposed on one side of a battery 350 along the short axis W and is located above the level, that is, above the long axis L. With reference to FIG. 6 first together with FIG. 2, an earbud 400 provided the present embodiment may be smoothly worn in the concha cavity 21 of the user as well, and the earbud 400 and the tragus 22 are located on the same level. Different from the foregoing embodiments, the sensor 130 provided by this embodiment is disposed on one side of a battery 450 along the short axis W and is located below the level, that is, below the long axis L. Based on the foregoing embodiments, it may be seen that the sensor 130 is adjacent to the battery 150 (250, 350, and 450) in the main body 110. That is, the sensor 130 may be appropriately disposed around the battery 150 (250, 350, and 450) except a position where the speaker 160 is adjacent to the battery 150 (250, 350, and 450).

In view of the foregoing, in the embodiments of the disclosure, the main body of the earbud is integrated with the battery and the sensor. Herein, the long axis and the short axis may be identified on the geometric shape of the main body, so that the tragus of the user and the long axis may be configured to be located on the same level when the earbud is worn. The sound guiding structure extends away from the main body from the long axis of the main body. Through the above features, the earbud may be securely housed in the concha cavity of the user. Further, the sensor which is disposed in the main body together with the battery is adjacent to the periphery of the battery. Therefore, through the foregoing arrangement, the earbud may not only set the sensor and the battery to be housed in the main body but also accordingly form a compact arrangement among members, and structural assembly may thus be smoothly completed.

Moreover, the sensing surface of the sensor protrudes from the inner side surface of the main body and thus faces away from the external environment and faces inwardly. As such, interference caused by external environment light may be effectively prevented, and heart rate of the user may be accurately sensed. Accordingly, through the above-mentioned structural features and member arrangement, the earbud may provide the required optical signal measurement effect, and the user may at the same time enjoy a comfortable wearing experience when wearing the earbud, so that the earbud may be worn for a long time and hear rate may be sensed at any time.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An earbud, comprising:
a main body, a geometric shape of the main body has a long axis and a short axis orthogonal to each other, wherein the main body is housed in a concha cavity of a user when the earbud is worn, and a tragus of the user and the long axis are located on a same level;
a sound guiding structure, extending away from the main body from the long axis of the main body;
a battery, disposed in the main body; and
a sensor, disposed in the main body, adjacent to a periphery of the battery.

2. The earbud according to claim 1, wherein a portion of the sensor protrudes from an inner side surface of the main body, the inner side surface faces a concha cavity wall of the user, and the sound guiding structure extends out from the inner side surface.

3. The earbud according to claim 1, wherein the sound guiding structure and the sensor are located on two opposite ends of the long axis, and the battery is located between the sound guiding structure and the sensor.

4. The earbud according to claim 3, wherein a position of the battery on the long axis and a position of the sensor on the long axis do not overlap with each other.

5. The earbud according to claim 1, wherein the position of the sensor on the long axis and a position of the battery on the long axis at least partially overlap, and an overlap is away from the sound guiding structure.

6. The earbud according to claim 5, wherein the sensor and the sound guiding structure are located on a same side of the battery, and a gap is provided between the battery and the sound guiding structure.

7. The earbud according to claim 1, wherein the sensor is disposed on one side of the battery along the short axis and is located above the level.

8. The earbud according to claim 1, wherein the sensor is disposed on one side of the battery along the short axis and is located below the level.

9. The earbud according to claim 1, further comprising a speaker disposed in the sound guiding structure.

10. The earbud according to claim 9, wherein the speaker is adjacent to the battery, and a position of the sensor on the long axis and a position of the battery on the long axis do not overlap with each other.

11. The earbud according to claim 9, wherein a gap is provided between the speaker and the battery, and a position of the sensor on the long axis and a position of the battery on the long axis at least partially overlap.

12. The earbud according to claim 1, wherein in a front view when the earbud is worn, an included angle is provided between an extending axis direction of the sound guiding structure and the long axis, and the included angle is 0 degrees to 45 degrees.

13. The earbud according to claim 1, wherein in a top view when the earbud is worn, an included angle is provided between an extending axis direction of the sound guiding structure and the long axis, and the included angle is 45 degrees to 80 degrees.

14. The earbud according to claim 1, wherein a ratio of the long axis to the short axis is: short axis/long axis=0.7 to 0.9.

15. The earbud according to claim 1, wherein a length of the short axis is 13.5 mm to 15.5 mm.
